(19) United States
(12) Patent Application Publication
(10) Pub. No.: US 2006/0258957 A1
(43) Pub. Date: Nov. 16, 2006

(54) Blood drawing system

(76) Inventors: Luis DeLeon, Chicago, IL (US); Jeime Aramburo, Chicago, IL (US)

Correspondence Address: BARNES & THORNBURG LLP, P.O. BOX 2786, CHICAGO, IL, 60690-2786, US

(21) Appl. No.: 11/431,200

(22) Filed: May 9, 2006

Related U.S. Application Data
(60) Provisional application No. 60/679,564, filed on May 10, 2005.

(51) Int. Cl. A61B 5/00

(52) U.S. Cl. 600/573

(57) ABSTRACT

A blood drawing system for drawing blood into evacuated containers and the like. A needle adapter is shaped for connection to a blood needle and includes a butterfly handle to guide vein puncture by the needle engaged in the adapter. A tube connector is provided for connection to a blood collection container, and a flexible tube extends between the adapter and the tube connector.

### BACKGROUND OF THE INVENTION

This invention relates to blood drawing, and in particular to a blood drawing system for use with multiple types of blood needles.

Blood is drawn from patients hundreds of thousands of time each day in the United States. Various blood drawing systems have been developed, but the most common is where, upon vein puncture, blood is drawn into evacuated tubes for later use and analysis.

Various types of such blood drawing systems have been developed in the past. One employs a butterfly needle, that is, a vein puncture needle having a butterfly-shaped handle which is well known as simply a butterfly. A typical butterfly is shown, for example, in U.S. Pat. No. 5,921,969. The wings of the butterfly are used by the medical practitioner for guiding the needle during vein puncture.

Another type of blood drawing system is shown in FIG. 1 of U.S. Pat. No. 4,841,985, employing a needle holder into which a double ended cannula or needle is threaded. An evacuated collection tube is used for blood collection, and multiple tubes can be used with the same needle and holder combination.

### SUMMARY OF THE INVENTION

The invention is directed to a blood drawing system, comprising a needle adapter with means for connection to a blood needle. A handle is located approximate the adapter, and a tube connector is provided for connection to a blood collection container. A flexible tube connects the adapter to the tube connector.

In accordance with the preferred form of the invention, the blood collection system employs a blood needle which is shaped lo be removably engaged in the needle adapter. The needle adapter comprises an open body having a conical inner configuration, and the connection means comprises a union on an entry port to the body. In accordance with the preferred form of the invention, the union is threaded, but also can be any type of connection to securely connect a blood needle into the needle adapter.

The conical inner configuration of the needle adapter includes an orifice shaped to conform to the size of needle extending therethrough. Thus, the conical shape guides the needle to a working position as the needle is engaged in the needle adapter.

The handle according to the invention is a butterfly-shaped grip which is secured to the adapter. The handle may be an integral part of the adapter, or can be a separate part that is appropriately affixed to the adapter to assist the practitioner in vein puncture.

### BRIEF DESCRIPTION OF THE DRAWING

The invention is described in greater detail in the following description of an example embodying the best mode of the invention, taking in conjunction with the drawing figures, in which:

FIG. 1 is an exploded elevational illustration of the blood drawing system according to the invention.

FIG. 2 is an enlarged cross sectional view of the needle adapter according to the invention.

FIG. 3 is an elevational view, slightly reduced in size, of a typical blood needle used in combination with the blood drawing system of FIG. 1.

### DESCRIPTION OF EXAMPLES EMBODYING THE BEST MODE OF THE INVENTION

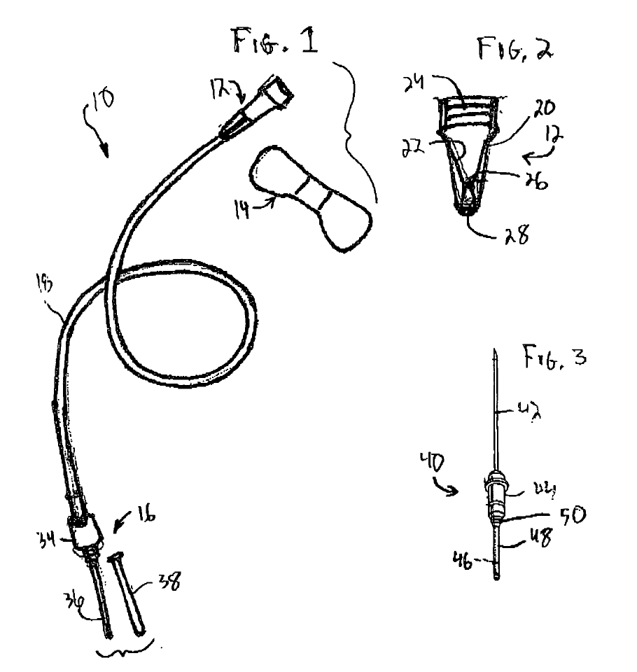

The blood drawing system according to the invention is shown generally at 10 in FIG. 1. The blood drawing system 10 comprises four basic elements, a needle adapter 12, a handle 14, a tube connector 16 and a flexible tube 18.

The needle adapter 12, as best shown in cross section in FIG. 2, comprises an open body 20 preferably having a conical inner configuration 22. At one ends the open body 20 includes a threaded union 24 for connection to a typical blood needle, or any other appropriate fitting or adapting structure used for connecting a blood needle to the needle adapter 12. The conical inner configuration terminates, at its smaller end, at an orifice 26 which is shaped to conform to a needle extending therethrough, as described in greater detail below. The orifice typically is about the same size as the needle, and is therefore sized depending an the needle to be extended therethrough, typically on the order of one sixteenth inch or so.

The orifice 26 then opens to a connecting orifice 28 sized and shaped to accommodate insertion of the flexible tube 18.

The handle 14 is a typical butterfly-shaped grip which is configured to be rigidly secured to the needle adapter 12. Securing can be by means of adhesives, sonic welding, force fitting, or any other means of rigidly, and essentially permanently, securing the handle 14 to the needle adapter 12. The handle 14 includes opposite butterfly wings 30 and 32 for guiding vein puncture in a conventional fashion, and the wings are therefore not further described.

The tube connector 16 can be a conventional connector at the end of the tube 18, and comprises a hollow union 34 having a needle 36 extending therefrom. The needle 36 is sized and shaped appropriately for insertion in a typical blood drawing container (not illustrated), such as an evacuated tube or the like, such as that illustrated in FIG. 1 of U.S. Pat. No. 4,841,985, the disclosure of which is incorporated herein by reference. A rigid sheath 38 is typically provided for covering the needle 36 until use in order to prevent inadvertent needle sticks. The tube connector 16 can be conventional, and is therefore not described in greater detail.

The flexible tube 18 is just that—a hollow, flexible tube of a desired length connecting the needle adapter 12 to the tube connector 14. Preferably, the flexible tube 18 is permanently installed in both the needle adapter 12 and the tube connector 16.

A typical blood needle 40 used in connection with the blood drawing system 10 of FIG. 1 is shown in FIG. 3. The blood needle 40 may be any one of a number of conventional blood needles, and is a double ended structure, having a vein puncture needle 42 connected to a hub 44 in communication with a second needle 46 covered by a displaceable prophylactic sheath 48. As is conventional, when the sheath 58 is retracted, as illustrated in FIG. 2 of incorporated U.S. Pat. No. 4,841,985, blood can then flow through the blood needle 40 during a blood draw. The hub 44 includes a male-threaded connector 50 shaped to he engaged within the threaded union 24.

In use, the blood drawing system 10 according to the invention is assembled as shown in FIG. 1 with the handle 14 secured to the needle adapter 12. The blood needle 40, which typically has rigid protectable covers (not illustrated) on both ends, is inserted by removing the rigid cover over the needle 46, allowing the blood needle 40 to be installed in the needle adapter 12. The conical inner configuration guides the needle 46 into the orifice 26, and as the blood needle 40 is installed, the sheath 48, which is much larger than the orifice 26, is retracted to allow the needle 46 to extend through the orifice 26.

Thereafter, the blood drawing system according to the invention is used for vein puncture in a conventional fashion. Any sheath over the needle 42 is withdrawn, and the patient's vein is then engaged and pierced with the needle 42. Blood then flows into the flexible tube 18, and connection of an evacuated container to the needle 36 then permits any number of evacuated containers to be filled for diagnostic and testing purposes.

Preferably the blood drawing system 10 according to the invention is disposable, and due to the universal nature of the needle adapter 12, can accommodate practically any type of conventional blood needle 40. Thus, unlike typical use of a blood needle 40 using a large and bulky holder, the system of the present invention allows a butterfly-like blood draw, without the use of a dedicated butterfly needle.

Various changes can be made to the invention without departing from the spirit thereof or scope of the following claims.

What is claimed is:

1. A blood drawing system, comprising:
a. a needle adapter having means for connection to a blood needle,
b. a handle located proximate said adapter,
c. a tube connector for connection to a blood collection container, and
d. a flexible tube connecting said adapter to said tube connector.

2. The blood connection system according to claim 1, including a blood needle shaped to be removably engaged in said needle adapter.

3. The blood connection system according to claim 1, in which said needle adapter comprises an open body having a guiding inner configuration, and said connection means comprising a union on an entry port to said body.

4. The blood connection system according to claim 3, in which said union is threaded.

5. The blood connection system according to claim 3, in which said guiding inner configuration is conical.

6. The blood connection system according to claim 3, in which said guiding inner configuration includes an orifice shaped to conform to a needle extending therethrough.

7. The blood connection system according to claim 1, in which said handle comprises a butterfly-shaped grip secured to said adapter.

8. A blood drawing system, comprising,
a. a needle adapter,
b. a blood needle shaped to be removably engaged in said needle adapter,
c. a handle located at said adaptor,
d. a tube connector for connection to a blood collection container, and
e. a flexible tube connecting said adapter to said tube connector.

9. The blood connection system according to claim 8, in which said needle adapter comprises an open body having a guiding inner configuration, and said connection means comprising a union on an entry port to said body.

10. The blood connection system according to claim 9, in which said union is threaded.

11. The blood connection system according to claim 9, in which said guiding inner configuration is conical.

12. The blood connection system according to claim 8, in which said guiding inner configuration includes an orifice shaped to conform to a needle extending therethrough.
